(19) United States
(12) Patent Application Publication
(10) Pub. No.: US 2015/0202145 A1
(43) Pub. Date: Jul. 23, 2015

(54) SUNSCREEN COMPOSITION WITH IMPROVED AESTHETIC PROPERTIES

(71) Applicant: MD SOLARCIENCES CORP., Norwalk, CT (US)

(72) Inventor: Robert Friedman, Stamford, CT (US)

(73) Assignee: MD SOLARCIENCES CORP., Norwalk, CT (US)

(21) Appl. No.: 14/159,954

(22) Filed: Jan. 21, 2014

(51) Int. Cl. A61K 8/97; A61K 8/58; A61K 8/27; A61K 8/67; A61Q 17/04; A61K 8/29

(52) U.S. Cl. CPC A61K 8/97; A61Q 17/04; A61K 8/29; A61K 8/27; A61K 8/676; A61K 8/585

(57) ABSTRACT

Sunscreen compositions having an SPF of 30 to 50 which include mixtures of zinc oxide and titanium dioxide, cross linked silicone-containing compounds and anti-oxidants to provide protection against UVA and UVB.

### BACKGROUND OF THE INVENTION

1. Field of the Invention

The present invention generally relates to sunscreen compositions that are suitable for topical application to human skin and hair. More particularly, the invention relates to aesthetically improved sunscreen compositions containing inorganic sunscreen agents.

2. Description of Related Art

The damaging effects of sunlight on human skin have long been noted. In general terms, harmful ultra-violet (UV) rays, particularly those originating from sunlight, which penetrate the upper atmosphere and reach the earth's surface can be classified into two types: (i) high energy UV-B rays (290-320 nm wavelength) which are absorbed just above the dermis and are responsible for sunburn and tanning effects; and (ii) low energy UV-A rays (320-400 nm wavelength) which penetrate deeper into the skin (to the dermis and beyond), and which cause damaging effects that are more long term in nature, such as skin ageing. Continued skin damage resulting from unprotected exposure to ultraviolet radiation can cause more serious conditions such as, for example, skin cancer. Depending on weather conditions, even casual unprotected exposure to the sun's ultraviolet radiation can be detrimental to one's skin. Accordingly, it is generally recommended that a sunscreen product be applied to the skin before exposure to ultraviolet radiation.

Sunscreen compositions or formulations may be applied directly to the skin to combat sunburning of the skin and the numerous damaging effects resulting from exposure to the sun. When a sunscreen formulation, utilizing the proper sun protection factor (SPF, which is a measure of the protection from the sun afforded by a sunscreen agent or composition) is applied uniformly to the body, sunscreens can be highly effective in protecting against sunburn and damage that can lead to photoaging.

Sunscreens containing inorganic particulates (titanium dioxide and zinc oxide) provide a good option for individuals with sensitive skin because these ingredients are not associated with irritation or sensitization. However, to the inventors' knowledge, prior formulations containing inorganic sunscreen have usually been pasty and thick and not to most people's liking. Therefore, there remains a need to develop novel methods and compositions for SPF sun protection.

### SUMMARY OF THE INVENTION

Aspects of the present invention overcome a major deficiency in the art by providing novel compositions that comprise inorganic sunscreen with improved aesthetic properties by the use of silicone-containing compounds. The use of metal oxides in prior sunscreen formulations has been limited due to their unappealing aesthetic properties. For example, inorganic sunscreen such as titanium dioxide or zinc oxide pose great difficulty in achieving ease of spreading, evenness of the application on skin, the dragginess of the filters, and the often white or “blue” hue given to the skin. Moderate SPF values of 15 and below can achieve quite nice aesthetic properties, with higher SPFs having less desirable aesthetic properties.

There is provided in an embodiment of the present invention a topically applicable cosmetic/dermatological high SPF-UV-screening composition comprising: (a) a sunscreen; (b) an aesthetic enhancer that comprises at least one cross-linked silicone elastomer and at least one other silicone-containing compound; (c) one or more antioxidants; and (d) a dermatologically acceptable vehicle. Such a composition may provide UVA-UVB protection that reduces the risk of photo aging and skin cancer.

In certain aspects, the sunscreen in accordance with the present invention may comprise inorganic sunscreen agents. They may be metal oxides, such as zinc oxide, titanium oxide, or a combination of both zinc oxide and titanium oxide.

The combination of zinc oxide and titanium oxide is preferred. In particular aspects, the weight percentage of the inorganic sunscreen or metal oxides can range from 0.1% to about 40%, by weight of the composition. The amount by weight of the inorganic sunscreen agent may range from about 0.1 to about 30%, by weight, or preferably from about 3% to about 25%, by weight of the composition.

Zinc oxide is the broadest spectrum UVA and UVB reflector that is approved for use as a sunscreen by the FDA and it is completely photostable. The zinc oxide in a sunscreen sits on the skin surface and is not absorbed into the skin and blocks both UVA (320-400 nm) and UVB (380-320 nm) rays of ultraviolet light. Zinc oxide and titanium dioxide are not absorbed into the skin, they are non-irritating, non-allergenic and non-comedogenic.

In accordance with the present invention, the weight percentage of zinc oxide may be from about 3% to about 40%, preferably from 20% to about 35%, and most preferably from 15% to about 17%. The weight percentage of titanium dioxide in the sunscreen composition may be from 1.0% to about 40%, by weight, of the composition, preferably from 1.0% to 10%, and most preferably from 1.5% to 3.0%.

In particular, the weight percentage of zinc oxide, may be in the range of 13.5% to 18.7% (w/w) and/or the weight percentage of titanium dioxide may be in the range of 1.8% to 3.3% (w/w). The ratio of zinc oxide to titanium oxide can be from about 1:1 to about 20:1, with a ratio of about 5:1 to about 12:1 being preferred.

A larger percentage of zinc oxide compared to titanium oxide is favored since the manufacturing technology for ZnO has improved significantly, and also from an aesthetic standpoint, it feels better and does not become white upon sweating or swimming.

The ratio of the aesthetic enhancer to all silicone-containing compounds to inorganic sunscreen or total metal oxides can be from 5:1 to about 10:1 based on the total weight of the composition.

It has been found advantageous to employ one or more antioxidants, preferably at least two (2) antioxidants, and most preferably at least four (4) antioxidants, although more than four (4) antioxidants could be used to advantage in the sunscreen composition of the present invention. In particular, antioxidants have demonstrated the ability to quench the free radicals associated with UVA radiation and thus provide protection against the occurrence of photo aging of the skin which results from the exposure to UVA rays. Since free radicals are said to account for about 90% of skin aging, including wrinkles, roughness, mottled pigmentation and loss of elasticity, the importance of incorporating antioxidants in sunscreens, sunblock cosmetics and toiletries as a defense against oxidative stress is readily apparent.

In certain aspects, the composition may further comprise at least one aesthetic enhancer that may be effective to improve aesthetic properties of the inorganic sunscreen agents. The aesthetic enhancer may comprise cross-linked silicone elastomers such as dimethicone crosspolymer and dimethicone/vinyl dimethicone crosspolymer. The aesthetic enhancer may comprise one or more silicone-containing compounds, such as dimethiconol, aromatic silicone, stearoxytrimethylsilane, alkylmethyl methicone, dimethicone copolyol, and polymethylsilsesquioxane. In certain aspects, the composition may comprise one, two, three, four, five, or all of the silicone-containing compounds referenced in the previous sentence. For example, the aromatic silicone may be phenyl methicone. An example of the dimethicone copolyol may be lauryl PEG/PPG-18 methicone. The composition may further comprise dimethicione. In exemplary embodiments, the aesthetic enhancer may comprise or consist essentially of dimethiconol, dimethicone, phenyl methicone, stearoxytrimethylsilane, and caprylyl methicone. In other exemplary embodiments, the aesthetic enhancer may comprise or consist essentially of dimethiconol, dimethicone, phenyl methicone, stearoxytrimethylsilane, lauryl PEG/PPG-18 methicone, and polymethylsilsesquioxane. In further embodiments, the composition may comprise polyglyceryl-3 polydimethylsiloxyethyl dimethicone and/or triehoxylsilyethyl polydimethylsiloxyethyl hexyl dimethicione. The concentrations of these ingredients can range from 0.00001 to 99% by weight or volume of the composition or any integer or range derivable therein as explained in other portions of this specification.

In certain aspects, the composition of the present invention may further comprise at least one aesthetic enhancer that may be effective to improve the aesthetic properties of the inorganic sunscreen agents. The aesthetic enhancer preferably comprises one or more cross-linked silicone elastomers, such as, for example dimethicone crosspolymer and dimethicone/vinyl dimethicone crosspolymer. The aesthetic enhancer also includes at least one other silicone-containing compound, preferably a linear silicone containing compound. Exemplary embodiments of the preferred linear silicone for use in the present invention are dimethicone, caprylyl methicone, triethoxy caprylylsilane, and mixtures thereof.

The sunscreen in accordance with an embodiment of the present invention may be a broad-spectrum sunscreen. “Broad-spectrum sunscreen,” as used herein, refers to sunscreen that block at least UV-A and UV-B rays. The compositions of the present invention can diffract or block a broad spectrum of electromagnetic radiation. For example, the compositions can block UV-A, UV-B, UV-C radiation and/or any combination thereof. For example, a composition can be designed to block UV-B radiation but not UV-A radiation. As discussed throughout this specification, a composition can be designed to block and allow a wide range of different electromagnetic ranges (including, for example, ranges within the UV-A, UV-B, UV-C radiation ranges). By way of example only, the compositions can be designed to block electromagnetic radiation having a wavelength of about 200 to about 400, 250 to about 350, 300 to about 325, 200 to about 290, 290 to about 320, or to about 760 to about 2,500 nm. In other aspects, the compositions can permit transmission of a predetermined wavelength range of electromagnetic radiation. Examples of electromagnetic radiation that is not diffracted can include radiation having a wavelength of about 321 to about 400, 290 to about 315, 309 to about 314, or 1660 to about 1900 nm. In certain embodiments, the composition may have an SPF value of the sunscreen or the composition of at least or about 30, 35, 40, 45 or 50.

In a particular embodiment, the composition may be anhydrous. In a further aspect, the composition may be essentially free of oil. In certain aspects, the composition may comprise water or oil of less than or about 0.1%, 0.5%, 1%, 2%, 5%, 10%, or any range derivable therein. The compositions of the present invention can be transparent. The compositions can also be formulated into a sunscreen composition that is applied to skin. The compositions can also be formulated to be spread or sprayed onto the skin. The compositions can be included into a vehicle. The vehicle can include an emulsion, a cream, a lotion, a solution, an anhydrous base, a gel, a spray, or an ointment. The vehicle can be a cosmetic vehicle. The compositions can also be included in a product. The product, in non-limiting embodiments, can be a skin sunscreen product, a skin care product, a sunless skin tanning product, paint, ink, a glass coating, glass, cloth, plastic, or eye glasses, or other products known to those of ordinary skill in the art or identified throughout this specification. In certain aspects, the composition may be formulated as a spray, a stick, an emulsion, a cream, a lotion, a solution, an anhydrous base, a gel, a spray, or an ointment.

Compositions of the present invention may include other beneficial agents and compounds such as, for example, diluents, acute or chronic moisturizing agents (including, e.g., humectants, occlusive agents, and agents that affect the natural moisturization mechanisms of the skin), emollients, preservatives, solvents, antioxidants, anti-irritants, vitamins, structuring agents, pharmaceutical ingredients, trace metals, anti-microbial agents, botanical extracts, fragrances, dyes and color ingredients, structuring agents, thickening agent (thickeners and gelling agents), and/or emulsifiers (see U.S. Pat. No. 6,290,938).

The viscosity of the composition in accordance with the present invention may be from about 40,000 cps to about 130,000 cps.

Also disclosed in the present invention is a method of protecting an object from ultraviolet radiation comprising applying on the surface of the object or in incorporating into the object the compositions of the present invention. The composition can be topically applied to the object. The object can be skin, hair, or fingernails (including human and animal skin, hair, or fingernails). In certain aspects, the composition can be formulated for application at least once, twice, three, four, five or more times a day to the skin. In other aspects, the composition is sprayed, spread, or rubbed onto the object. The composition in certain embodiments, can be incorporated into the object. The object, by way of example only, can be any article of manufacture known to those of skill in the art or identified in this specification. For example, the object can be paint, ink, windows, self adhesive tap, eye wear (including eye glasses and contact), cloths (including clothing, car covers, boat covers), wood, protective coatings (e.g., water sealers, stains, etc.) or plastics.

Another aspect of the present invention discloses a method of making a composition comprising a sunscreen and an aesthetic enhancer, wherein the aesthetic enhancer may include a plurality of silicone-containing compounds, the method comprising (i) obtaining a plurality of silicone-containing compounds; (ii) obtaining a sunscreen; and (iii) admixing (i) and (ii), wherein the admixture is formulated into a composition. For example, the admixing may comprise heating the mixture at a temperature about or higher than room temperature, such as at least or about 20 to about 100° C., or any range derivable therein. Also, for example, the mineral lotions having an SPF of 30, 40 and 50 in accordance with the invention can, if desired, be made into a so-called “soufflé” by subjecting it to homogenization. This procedure can be followed with the SPF 50 mineral lotion described in Example 3. Souffles are particularly gentle to the skin.

Also disclosed is a kit comprising the compositions of the present invention. The compositions can be included in a container. In non-limiting aspects, the container can be a bottle, a dispenser, or a package. In certain embodiments, the container can dispense a pre-determined amount of the composition. The composition can be dispensed in a spray, an aerosol, or in a liquid form or semi-solid form. In certain aspects, the container can include indicia on its surface. The indicia, for example, can be a word, a phrase, an abbreviation, a picture, or a symbol. The word or phrase can be “sunscreen,” “sunblock,” “UV specific sunblock,” etc.

In another embodiment, there is disclosed a product or article of manufacture comprising the compositions of the present invention. Product and articles of manufacture that are contemplated as being useful with the present invention are those known to a person of ordinary skill in the art and those identified in this specification. Non-limiting examples include sunscreen products, sunblock products, cosmetic products (e.g., sunless tanning product, moisturizers, creams, lotions, skin softeners, foundations, night creams, lipsticks, cleansers, toners, masks, and other make-up products), paint, ink, cloths (e.g., clothing, tarps, car and boat covers, ext.), glass, glass films, eye ware (e.g., eye glasses and contacts), coatings, windows, plastics, ext.

Embodiments discussed in the context of methods and/or compositions of the invention may be employed with respect to any other method or composition described herein. Thus, an embodiment pertaining to one method or composition may be applied to other methods and compositions of the invention as well.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The use of sunblock compositions have gained more and more popularity over the years. For example, sunblock compositions can be used to protect a person's skin, hair, finger nails, or an article of manufacture from the sun's or artificial electromagnetic radiation. A problem associated with previous sunblock compositions using inorganic sunscreen, however, is their inability to provide pleasant aesthetic properties, especially while having high SPF values (more than 30) contributed by high concentration of inorganic sunscreen such as zinc oxide or titanium oxide. In the second embodiment of the present invention, the SPF value can be about 40 or above or even 50 or above.

In accordance with certain aspects of the present invention, there are provided compositions that have several advantages over previous compositions. The disclosed compositions, in non-limiting aspects, include an aesthetic enhancer that may include a plurality of silicone-containing compounds that are effective to improve aesthetic properties of the inorganic sunscreen agents. The composition in non-limiting aspects, includes at least one antioxidant and preferably two or more antioxidants. However, even four (4) or more antioxidants can be used to advantage. The compositions can be used to protect, for example, a person's skin, hair, finger nails, or an article of manufacture from damaging electromagnetic radiation such as UV.

These and other aspects of the present invention are described in further detail in the following sections.

### I. SUNSCREEN AGENTS

There are currently seventeen (17) active ingredients approved by the FDA for use in sunscreens. These filters fall into two broad categories: organic/chemical and inorganic/physical. Most UV filters are organic: They form a thin, protective film on the surface of the skin and absorb the UV radiation before it penetrates the skin. The inorganic sunscreens are insoluble particles that reflect UV away from the skin. Most current sunscreens contain a mixture of organic and inorganic active ingredients.

UV absorption agents that can be used in combination with the compositions of the present invention include at least inorganic sunscreen. Non-limiting examples of inorganic sunscreen include metal oxides (e.g., titanium dioxide and zinc oxide). The compositions can have an sun protection factor (SPF) of 2, 3, 4, 5, 6, 7, 8, 9, 10, 11, 12, 13, 14, 15, 20, 25, 30, 35, 40, 45, 50, 55, 60, 70, 80, 90 or more, or any integer or derivative therein.

The sunscreen used in the present invention may further comprise an organic sunscreen. Non-limiting examples of organic sunscreen agents that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloy trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutyiphenol, and bis-ethylhexyloxyphenol methoxyphenyltriazine, 4-methylbenzylidenecamphor, and isopentyl 4-methoxycinnamate.

Compositions of the present invention can have UV-A and UV-B protection properties. For example, the composition can comprise one or more sunscreen agents that have UV-A protection properties, one or more sunscreen agents that have UV-B protection properties, and/or one or more sunscreen agents that have both UV-A and UV-B protection properties. Table 1 provides information on known UV-A or UV-B protection properties of FDA-approved sunscreens.

TABLE 1
FDA-Approved Sunscreens

FDA-Approved Sunscreens
Active Ingredient/UV Filter Name
Chemical/organic absorbers:

| | |
|---|---|
| Aminobenzoic acid (PABA) | UV-B |
| Avobenzone | UV-A1 |
| Cinoxate | UV-B |
| Dioxybenzone | UV-B, UV-A2 |
| Ensulizole (Phenylbenzimiazole Sulfonic Acid) | UV-B |
| Homosalate | UV-B |
| Meradimate (Menthyl Anthranilate) | UV-A2 |
| Octocrylene | UV-B |
| Octinoxate (Octyl Methoxycinnamate) | UV-B |
| Octisalate (Octyl Salicylate) | UV-B |
| Oxybenzone | UV-B, UV-A2 |
| Padimate O | UV-B |
| Sulisobenzone | UV-B, UV-A2 |
| Trolamine Salicylate | UV-B |
| Titanium Dioxide | UV-B, UV-A2 |
| Zinc Oxide | UV-B, UV-A2, UV-A1 |

Range Covered
UV-A1: 340-400 nm
UV-A2: 320-340 nm
UV-B: 290-320 nm

For a product marketed in the United States, preferred cosmetically-acceptable photoactive compounds and concentrations (reported as a percentage by weight of the total cosmetic sunscreen composition) include: aminobenzoic acid (also called para-aminobenzoic acid and PABA; 15% or less), avobenzone (also called butyl methoxy dibenzoylmethane; 3% or less), cinoxate (also called 2-ethoxyethylp-methoxycinnamate; 3% or less), dioxybenzone (also called benzophenone-8; 3% or less), homosalate (15% or less), menthyl anthranilate (also called menthyl 2-aminobenzoate; 5% or less), octocrylene (also called 2-ethylhexyl-2-cyano-3,3 diphenylacrylate; 10% or less), octyl salicylate (also called 2-ethylhexyl salicylate; 5% or less), padimate 0 (also called octyl dimethyl PABA; 8% or less), phenylbenzimidazole sulfonic acid (water soluble; 4% or less), sulisobenzone (also called benzophenone-4; 10% or less), titanium dioxide (25% or less), trolamine salicylate (also called triethanolamine salicylate; 12% or less), and zinc oxide (25% or less).

For a product marketed in the European Union, preferred cosmetically-acceptable photoactive compounds and preferred concentrations (reported as a percentage by weight of the total cosmetic sunscreen composition) include: PABA (5% or less), camphor benzalkonium methosulfate (6% or less), homosalate (10% or less), benzophenone-3 (10% or less), phenylbenzimidazole sulfonic acid (8% or less, expressed as acid), terephthalidene dicamphor sulfonic acid (10% or less, expressed as acid), butyl methoxydibenzoylmethane (5% or less), benzylidene camphor sulfonic acid (6% or less, expressed as acid), octocrylene (10% or less, expressed as acid), polyacrylamidomethyl benzylidene camphor (6% or less), ethylhexyl methoxycinnamate (10% or less), PEG-25 PABA (10% or less), isoamyl p-methoxycinnamate (10% or less), ethylhexyl triazone (5% or less), drometrizole trielloxane (15% or less), diethylhexyl butamido triazone (10% or less), 4-methylbenzylidene camphor (4% or less), 3-benzylidene camphor (2% or less), ethylhexyl salicylate (5% or less), ethylhexyl dimethyl PABA (8% or less), benzophenone-4 (5%, expressed as acid), methylene bis-benztriazolyl tetramethylbutylphenol (10% or less), disodium phenyl dibenzimidazole tetrasulfonate (10% or less, expressed as acid), bis-ethylhexyloxyphenol methoxyphenol triazine (10% or less), methylene bisbenzotriazolyl tetramethylbutylphenol (10% or less, also called TINOSORB M), and bisethylhexyloxyphenol methoxyphenyl triazine. (10% or less, also called TINOSORB S).

### II. SILICONE CONTAINING COMPOUNDS

In certain aspects of the invention, an asthetic enhancer comprising a plurality of silicone-containing compounds may be used for the unexpected enhancement of asthetic properties of the inorganic sunscreen such as metal oxides comprised in the composition.

In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

1. Crosslinked Silicone Elastomer

The present composition may include one or more crosslinked silicone elastomers. An elastomer is generally a chain polymer having a degree of crosslinking sufficient to provide a rubberlike consistency. A crosslinked silicone elastomer is formed from a siloxane polymer having at least two free reactive groups preferably bonded to at least one terminal silicon atom. The reactive groups can include, but are not limited to, vinyl, allyl or epoxide moieties. The groups then react with Si—H linkages of another suitable polysiloxane backbone capable of participation in an addition reaction, such as a molecularly spherical MQ resin. The reaction results in a three-dimensional crosslinked polymer. The crosslinked silicone elastomer is essentially non-emulsifying (e.g., polyoxyalkylene groups absent). The average molecular weight of the crosslinked silicone elastomer preferably ranges from about 2,000 to about 20 million, most preferably from about 1 million to about 20 million.

Examples of crosslinked silicone elastomers suitable for use in the present compositions include, but are not limited to, one or more dimethicone crosspolymers, such as DC 9040 of Dow Corning Corp.; organopolysiloxanes (a.k.a. polysilicone-11), such as GRANSIL GCM and GRANSIL SR-5CYC of Grant Industries; dimethicone/vinyl dimethicone crosspolymers, such as SFE839 of General Electric, KSG15 of Shin-Etsu, and DC 9509 of Dow Corning Corp.; or any combinations thereof. Dimethicone/vinyl dimethicone crosspolymers for use in the present compositions include those disclosed in U.S. Pat. Nos. 5,871,761 and 6,013,682, which are incorporated herein by reference. Some examples of such useful crosslinked silicone elastomers are those provided in the form of non-ionic suspensions (for example, DC 9546 SILICONE ELASTOMER BLEND or DC 9509 SILICONE ELASTOMER SUSPENSION of Dow Corning Corp.) or non-ionic emulsions (for example, DC HMW 2220 NON-IONIC EMULSION of Dow Corning Corp.).

It is preferred that the crosslinked silicone elastomer is solid. By the term “solid” it is meant that the crosslinked silicone elastomer does not flow or have a viscosity as do silicone fluids. DC 9509 from Dow Corning Corp., discussed above, is an example of such a solid crosslinked silicone elastomer.

Although crosslinked silicone elastomers can be added directly to the composition, it is preferred that the crosslinked silicone elastomer is added to the composition in the form of a pre-solubilized/pre-dispersed suspension or emulsion. When such a predispersed crosslinked silicone elastomer is used, as is most preferred, the amount of emulsifier/surfactant in the crosslinked silicone elastomer dispersion will be calculated as part of the level of emulsifier/surfactant contained in the overall sun protection composition, as will be discussed further below.

The crosslinked silicone elastomer is present in the composition in an amount effective to decrease sebum on the skin or hair as compared to a composition without the crosslinked silicone elastomer. For example, the crosslinked silicone elastomer is present at about 0.01 wt % to about 40 wt %, based on the total weight of the composition. More particularly, the crosslinked silicone elastomer is present at about 0.1 wt % to about 30 wt %, based on the total weight of the composition. Most particularly, the crosslinked silicone elastomer is present at about 0.5 wt % to about 25 wt %, based on the total weight of the composition.

2. Other Silicone-Containing Compounds

The silicone containing compounds that can be used in the context of the present invention include those described in this specification or those known to a person of ordinary skill in the art. Non-limiting examples include silicone oils (e.g., volatile and non-volatile oils), gels, and solids. In preferred aspects, the silicon containing compounds includes a silicone oils such as a polyorganosiloxane. Non-limiting examples of polyorganosiloxanes include dimethicone, dimethiconol, alkylmethyl methicone, dimethicone copolyol, cyclomethicone, polysilicone-11, phenyl trimethicone, trimethylsilylamodimethicone, stearoxytrimethylsilane, polymethylsilsesquioxane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich.

The use of one or more straight chain silicones such as dimethicone, caprylyl methicone, triethoxy caprylsilane, and mixtures thereof, have been found to be particularly advantageous for use in accordance with the present invention.

### III. COMPOSITIONS OF THE PRESENT INVENTION

1. Combinations and Amounts of Ingredients

It is contemplated that the compositions of the present invention can include an aesthetic enhancer comprising a plurality of silicone-containing compounds. The compositions can also include additional ingredients described throughout this specification. The concentrations of the silicone-containing compounds and/or additional ingredients can vary. In non-limiting embodiments, for example, the compositions can include in their final form, for example, at least about 0.0001% to about 99% or more, or any range or integer derivable therein, of at least one of the silicone-containing compounds identified in this specification or any combination thereof or additional ingredients. In non-limiting aspects, the percentage of such ingredients can be calculated by weight or volume of the total weight of the compositions. The concentrations can vary depending on the desired effect of the compositions or on the product into which the compositions are incorporated.

2. Composition Vehicles

The compositions of the present invention can be formulated into all types of vehicles. Non-limiting examples of suitable vehicles include emulsions (e.g., oil-in-water, water-in-oil, silicone-in-water, water-in-silicone, water-in-oil-in-water, oil-in-water, oil-in-water-in-oil, oil-in-water-in-silicone, etc.), creams, lotions, solutions (both aqueous and hydro-alcoholic), anhydrous bases (such as lipsticks and powders), gels, ointments, pastes, milks, liquids, aerosols, solid forms, or eye jellies. Variations and other appropriate vehicles will be apparent to the skilled artisan and are appropriate for use in the present invention. In certain aspects, the concentrations and combinations of the ingredients can be selected in such a way that the combinations are chemically compatible and do not form complexes which precipitate from the finished product.

It is also contemplated that the silicone-containing compounds and additional ingredients identified throughout this specification can be encapsulated for delivery to a target area such as skin. Non-limiting examples of encapsulation techniques include the use of liposomes, vesicles, and/or nanoparticles that can be used as delivery vehicles to deliver such ingredients to skin (see, e.g., U.S. Pat. No. 6,387,398; U.S. Pat. No. 6,203,802; U.S. Pat. No. 5,411,744; Kreuter 1988).

Also contemplated are pharmaceutically-acceptable or pharmacologically-acceptable compositions. The phrase “pharmaceutically-acceptable” or “pharmacologically-acceptable” includes compositions that do not produce an allergic or similar untoward reaction when administered to a human. Typically, such compositions are prepared either as topical compositions, liquid solutions or suspensions, solid forms suitable for solution in, or suspension in, liquid prior to use can also be prepared. Routes of administration can vary with the location and nature of the condition to be treated, and include, e.g., topical, inhalation, intradermal, transdermal, parenteral, intravenous, intramuscular, intranasal, subcutaneous, percutaneous, intratracheal, intraperitoneal, intratumoral, perfusion, lavage, direct injection (e.g., an injectable solution), and oral administration and formulation (e.g., tablets, capsules, etc.).

3. Products

The compositions of the present invention can be incorporated into products. Non-limiting examples of products include cosmetic products, food-based products (e.g., fortified water, energy drinks, nutritional drinks, vitamins, supplements, solid foods), pharmaceutical products, etc. By way of example only, non-limiting cosmetic products include sunscreen products, sunless skin tanning products, hair products (e.g., shampoos, conditioners, colorants, dyes, bleaches, straighteners, and permanent wave products), fingernail products, moisturizing creams, skin creams and lotions, softeners, day lotions, gels, ointments, foundations, night creams, lipsticks and lip balms, cleansers, toners, masks, deodorants, antiperspirants, exfoliating compositions, shaving-related products (e.g., creams, “bracers” and aftershaves), pre-moistened wipes and washcloths, tanning lotions, bath products such as oils, foot care products such as powders and sprays, skin colorant and make-up products such as foundations, blushes, rouges eye shadows and lines, lip colors and mascaras, baby products (e.g., baby lotions, oils, shampoos, powders and wet wipes), and skin or facial peel products. Additionally, the cosmetic products can be formulated as leave-on or rinse-off products.

4. Additional Ingredients

Compositions of the present invention can include additional ingredients. Non-limiting examples of additional ingredients include cosmetic ingredients (both active and non-active) and pharmaceutical ingredients (both active and non-active).

a. Cosmetic Ingredients

The CTFA International Cosmetic Ingredient Dictionary and Handbook (2008), 12^{th} Edition, describes a wide variety of non-limiting cosmetic ingredients that can be used in the context of the present invention. Examples of these ingredient classes include: fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, emulsifiers, stabilizers, lubricants, solvents, moisturizers (including, e.g., emollients, humectants, film formers, occlusive agents, and agents that affect the natural moisturization mechanisms of the skin), water-repellants, essential oils, vitamins (e.g., A, B, C, D, E, and K), trace metals (e.g., zinc, calcium and selenium), anti-irritants (e.g., steroids and non-steroidal anti-inflammatories), botanical extracts (e.g., aloe vera, chamomile, cucumber extract, ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., glycerin, propylene glycol, butylene glycol, pentylene glycol, sorbitol, urea, and manitol), exfoliants (e.g., alpha-hydroxyacids, and beta-hydroxyacids such as lactic acid, glycolic acid, and salicylic acid; and salts thereof) waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate), thickening agents (e.g., substances which that can increase the viscosity of a composition such as carboxylic acid polymers, crosslinked polyacrylate polymers, polyacrylamide polymers, polysaccharides, and gums), and silicone containing compounds (e.g., silicone oils and polyorganosiloxanes). The following provides specific non-limiting examples of some of the additional ingredients that can be used with the compositions of the present invention.

i. Moisturizing Agents

Non-limiting examples of moisturizing agents, sometimes also known as hydrators, that can be used with the compositions of the present invention include amino acids, caprylylglycol, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

ii. Antioxidants

The incorporation of at least one antioxidant or preferably several antioxidants into an organic-based sunscreen cosmetic/cosmeceutical has been found to protect the skin again UVA radiation (aging) and UVB radiation (burning).

Non-limiting examples of antioxidants that can be used with the compositions of the embodiments of present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea (camellia sinensis) or grape seed extracts, nordihydroguaiaretic acid, octyl gallate, phenylthioglycolic acid, potassium ascorbyl tocopheryl phosphate, potassium sulfite, propyl gallate, punica granatum (pomegranate) extract, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite and vaccinium macrocanpon (cranberry) fruit extract.

iii. Structuring Agents

In other non-limiting aspects, the compositions of the present invention can include a structuring agent. Structuring agents, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

iv. Emulsifiers

In some non-limiting aspects, the compositions can include one or more emulsifiers. Emulsifiers can reduce the interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

v. Essential Oils

Essential oils include oils derived from herbs, flowers, trees, and other plants. Such oils are typically present as tiny droplets between the plant's cells, and can be extracted by several methods known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant in which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present invention.

vi. Thickening Agents

Thickening agents, including thickener or gelling agents, include substances that can increase the viscosity of a composition. Thickeners include those that can increase the viscosity of a composition without substantially modifying the efficacy of the active ingredient within the composition. Thickeners can also increase the stability of the compositions of the present invention.

Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, 4^{th} Ed., 1991). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

b. Pharmaceutical Ingredients

Pharmaceutical ingredients are also contemplated as being useful with the emulsion compositions of the present invention. Non-limiting examples of pharmaceutical ingredients include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, Co-Q 10, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

### IV. KITS

Kits are also contemplated as being used in certain aspects of the present invention. For instance, a composition of the present invention can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of a composition. In other embodiments, the container can be squeezed (e.g., metal, laminate, or plastic tube) to dispense a desired amount of the composition. The composition can be dispensed as a spray, foam, an aerosol, a liquid, a fluid, or a semi-solid. The containers can have spray, pump, or squeeze mechanisms. A kit can also include instructions for using the kit and/or compositions. Instructions can include an explanation of how to apply, use, and maintain the compositions.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### An Example of SPF 30 Mineral Tinted Cream

Table 1 below includes a non-limiting example of a composition of the present invention (e.g., SPF 30 cream) (WT:weight). The composition can be formulated into a cream. The additional ingredient identified throughout the specification can be included in the Table 1 composition (e.g., by adjusting the emollient content of the composition). Further, the concentration range of the ingredients identified in Table 1 can vary depending on a desired formulation (e.g., cream, lotion, moisturizer, cleanser, etc.). Table 1 provides a specification of the composition tested in Table 1A. In this example, the formulation may have 17.00% by weight of zinc oxide and 2.00% by weight of titanium dioxide.

TABLE 1
Formulation for Mineral Tinted SPF 30 Cream

| No. | Phase | Ingredient (Trade Name) | Function | Supplier | % by wt |
|---|---|---|---|---|---|
| 1 | A | DC 9041 | Emulsifier | Dow | 58.92% |
| | | dimethicone and | | Corning | |
| | | dimethicone | | | |
| | | cross-polymer | | | |
| 2 | A | Zinclear IM | Sunscreen | Dow | 34.00 |
| | | Caprylic/Capric Triglyceride | | Chemical | |
| | | Glyceryl isostearate | | | |
| | | Polyhyroxy stearic acid | | | |
| | | Zinc oxide dispersion | | | |
| 3 | A | DC9701 | Cosmetic | Dow | 0.3% |
| | | dimethicone/ | Powder | Corning | |
| | | vinyldimethicone | | | |
| | | cross polymer | | | |
| | | and silica | | | |
| 4 | A | Butyloctyl salicylate | Sunscreen | HallStar | 2.30% |
| 5 | A | Parsol SLX | Sunscreen | DSM | 1.00% |
| | | Polysilicone-15 | | | |
| 6 | A | Parsol TX | Sunscreen | DSM | 2.40% |
| | | silica, dimethicone, titanium dioxide | | | |
| 7 | A | BVOCS Tetrahexyldecyl Ascorbate | Antioxidant | Barnett | 0.10% |
| | | | | Products | |
| 8 | A | Extract Blend | Antioxidants | Barnett | 0.10% |
| | | Camellia Simensis (Green Tea | | Prod. Corp. | |
| | | Extract) | | | |
| | | Vaccinium Marcrocarpon | | | |
| | | (Cranberry) Fruit Extract | | | |
| | | Punica Granatum (Pomegranate) | | | |
| | | Extract | | | |
| 9 | B | FAND55 RSI | Pigment | Kobo | 0.14% |
| | | Iron Oxides, Dimethicone, | Dispersion | Products | |
| | | and PEG/PPG-18/18 Dimethicone, | Emulsifier | | |
| | | Triethoxycaprylylsilane | | | |
| 10 | B | FAND45YSI | Pigment | Kobo | 0.6400% |
| | | Iron oxides | Dispersion | Products | |
| | | Dimethicone | Emulsifier | | |
| | | PEG/PPG - 18/18 Dimethicone | | | |
| | | Triethoxycaprylsilane | | | |
| 11 | B | FAND60BSI | Pigment | Kobo | 0.100% |
| | | Iron Oxides | Dispersion | Products | |
| | | Dimethicone | Emulsifier | | |
| | | PEG/PPG- 18/18 Dimethicone | | | |
| | | Triethoxycaprylylsilane | | | |
| | | | | TOTAL | 100.00 |

MANUFACTURING INSTRUCTIONS:

| MANUFACTURING | |
|---|---|
| INSTRUCTIONS: | |
| PHASE A | MIX PHASE A INGREDIENTS UNTIL UNIFORM |
| PHASE B | COMBINE PHASE B INGREDIENTS AND MIX UNTIL UNIFORM |
| | SLOWLY ADD PHASE A TO PHASE B IN INCREMENTS. MAKE |
| | SURE BATCH IS UNIFORM BEFORE ADDING NEXT |
| | INCREMENT |

TABLE 1A
Product Control Specification (SPF 30+ mineral tinted cream)

| TEST | SPECIFICATIONS | RESULTS |
|---|---|---|
| APPEARANCE | BROWN CREAM | PASS- MATCH STD. |
| ODOR | TO MATCH STD. | PASS - MATCH |
| | | STD. |
| SPECIFIC GRAVITY | 0.95-1.15 | 1.11 |
| VISCOSITY | 50,000-200,000 CPS | 101,000 CPS |
| (SPINDLE TE/10 RPM/ | | |
| 1 MIN) | | |
| ASSAY: Ti0₂ (2.0%) | 1.8%-2.2% | 1.94% |
| ASSAY: ZnO (17.0%) | 15.3%-18.7% | 17.48% |
| BACTERIOLOGY | LESS THAN 10 CFU | PASS - LESS THAN |
| | MOLD/G | 10 CFU - NO MOLD |
| | BACTERIAL G | NO PATHOGENS |
| | NO PATHOGENS | |

### Example 2

### An Example of SPF 50 Mineral Cream

Table 2 below includes a non-limiting example of the present invention (e.g., SPF 50) (WT: weight). The composition can be formulated into a cream. The additional ingredients identified throughout the specification can be included into the Table 2 composition (e.g., by adjusting the emollient content of the composition.) Further, the concentration range of the ingredients identified in Table 2 can vary depending on a desired formulation (e.g., cream, lotion, moisturizer, cleanser, etc.). Table 2 provides a specification of the composition tested in Table 2A. In this example, the formulation may have zinc oxide of about 17.0% by wt. and titanium oxide of about 2.0%, by wt.

TABLE 2
Formulation for SPF 50 Mineral Cream

| No. | Phase | Ingredient (Trade Name) | Function | Supplier | % by wt |
|---|---|---|---|---|---|
| 1 | A | DC 9041 | Emulsifier | Dow | 59.78% |
| | | dimethicone and | | Corning | |
| | | dimethicone | | | |
| | | cross-polymer | | | |
| 2 | A | Zinclear IM | Sunscreen | Dow | 34.00% |
| | | Caprylic/Capric | | Chemical | |
| | | Triglyceride | | | |
| | | Glyceryl isostearate | | | |
| | | Polyhyroxy stearic acid | | | |
| | | Zinc oxide dispersion | | | |
| 3 | A | DC9701 | Cosmetic | Dow | 0.3% |
| | | dimethicone/ | Powder | Corning | |
| | | vinyldimethicone | | | |
| | | cross polymer | | | |
| | | (and) silica | | | |
| 4 | A | Butyloctyl salicylate | Sunscreen | HallStar | 2.30% |
| | | | Emollient | | |
| 5 | A | Parsol SLX | Sunscreen | DSM | 1.00% |
| | | Polysilicone-15 | | | |
| 6 | A | Parsol TX | Sunscreen | DSM | 2.40% |
| | | silica | | | |
| | | dimethicone | | | |
| | | titanium dioxide | | | |
| 7 | A | BVOCS | Antioxidant | Barnett | 0.10% by |
| | | Tetrahexyldecyl | | Products | wt. |
| | | Ascorbate | | | |
| 8 | A | Extract Blend | Antioxidants | Barnett | 0.10% |
| | | Camellia Sinensis | | Products | |
| | | (Green Tea Extract) | | Corp. | |
| | | Vaccinium | | | |
| | | Marcrocarpon | | | |
| | | (Cranberry) Fruit | | | |
| | | Extract | | | |
| | | Punica Granatum | | | |
| | | (Pomegranate) Extract | | | |
| 9 | B | FAND55 RSI | Pigment | Aston | 0.0075% |
| | | Dimethicone, | Dispersion | Chemicals, | |
| | | PEG/PPG-18/18 | | Kobo | |
| | | Dimethicone | | Chemical | |
| | | Triethoxycaprylylsilane | | | |
| 10 | B | FAND45YSI | Pigment | Aston | 0.0075% |
| | | Iron oxides | Dispersion | Chemical | |
| | | Dimethicone | | Kobo | |
| | | PEG/PPG - 18/18 | | Chemical | |
| 11 | B | FAND60BSI | Pigment | Aston | 0.006% |
| | | Dimethicone | Dispersion | Chemical | |
| | | PEG/PPG- 18/8 | | Kobo | |
| | | Dimethicone | | Chemical | |
| | | Triethoxycaprylylsilane | | | |
| | | | | | TOTAL |
| | | | | | 100.00% |

MANUFACTURING INSTRUCTIONS:

| | |
|---|---|
| PHASE | MIX PHASE A INGREDIENTS UNTIL |
| A | UNIFORM |
| PHASE | COMBINE PHASE B INGREDIENTS AND MIX UNTIL UNIFORM |
| B | |
| | SLOWLY ADD PHASE A TO PHASE B IN INCREMENTS. MAKE SURE |
| | BATCH IS UNIFORM BEFORE ADDING NEXT INCREMENT |

TABLE 2A
Product Control Specification (SPF 50 cream 2)

| TEST | SPECIFICATIONS | RESULTS |
|---|---|---|
| APPEARANCE | OFF WHITE TO | PASS - MATCH |
| | LIGHT BEIGE | STD. |
| ODOR | TO MATCH STD. | PASS - MATCH |
| | | STD. |
| SPECIFIC GRAVITY | 0.95-1.15 | 1.11 |
| VISCOSITY | 50,000-200,000 CPS | 117,500 CPS |
| (SPINDLE TE/10 RPM/ | | |
| 1 MIN) | | |
| % SUNSCREENS | 1.8%-2.2% | 1.97% |
| (ASSAY: TiO₂) | | |
| % SUNSCREENS | 15.3%-18.7% | 16.55% |
| (ASSAY: ZnO) | | |
| BACTERIOLOGY | LESS THAN 10 CFU | PASS - LESS THAN |
| | MOLD/G, | 10 CFU - NO MOLD |
| | BACTERIA/G | AND NO |
| | NO PATHOGENS | PATHOGENS |

### Example 3

### An Example of SPF 50 Mineral Lotion

Table 3 below includes a non-limiting example of a composition of the present invention (e.g., SPF 50+ lotion) (WT: weight). The composition can be formulated into a lotion or any anhydrous formulation. The additional ingredients identified throughout the specification can be included in the Table 3 composition (e.g., by adjusting the emollient content of the composition). Further, the concentration range of the ingredients identified in Table 3 can vary depending on a desired formulation (e.g., cream, lotion, moisturizer, cleanser, etc.) Table 3 provides a specification of the composition which is tested in Table 3A. In this example, the formulation may have 15.00% by weight of zinc oxide and 3.00% by weight of titanium oxide.

TABLE 3
SPF 50 Mineral Lotion

| No. | Phase | Ingredient (Trade Name) | Function | Supplier | % by wt |
|---|---|---|---|---|---|
| 1 | A | KSG210 | Emulsifier | Shin Etsu | 5% |
| | | dimethicone and | | | |
| | | dimethicone PEG- | | | |
| | | 10/15 | | | |
| | | cross-polymer | | | |
| 2 | A | KF6017 | Emulsifier | Aston | 1% |
| | | PEG-10 Dimethicone | | Chemicals | |
| 3 | A | DC9041 | Emulsifier | Dow | 23.4% |
| | | dimethicone/and | | Corning | |
| | | dimethicone | | | |
| | | cross polymer | | | |
| 4 | A | TiO₂ | Sunscreen | Resources of | 3.0% |
| | | | Emollient | Nature | |
| 5 | A | Distinctive Gel OD | Quaternium-90 | Resources of | 5% |
| | | | Bentonite and | Nature | |
| | | | Isodecane and | | |
| | | | Propylene | | |
| | | | carbonate | | |
| 6 | A | Butyloctyl salicylate | Sunscreen | | |
| | | | Emollient | | |
| 7 | A | Extract Blend | Antioxidant | Barnett | 0.10% by |
| | | Tetrahexyldecyl | | Products | wt. |
| | | Ascorbate | | | |
| | | Camellia Simensis | | | |
| | | (Green Tea Extract) | | | |
| | | Vaccinium | | | |
| | | Marcrocarpon | | | |
| | | (Cranberry) Fruit | | | |
| | | Extract | | | |
| | | Punica Granatum | | | |
| | | (Pomegranate) Extract | | | |
| | | Tetrahexyldecyl | | | |
| | | Ascorbate (Vitamin C) | | | |
| 8 | A | BVOS-C | Antioxidant | | 1.0% |
| | | Tetrahyxyldecyl | | | |
| | | ascorbate | | | |
| 9 | A | Parsol SLX | Sunscreen | DSM | 1.0% |
| | | Polysilicone 15 | | | |
| 10 | B | Water | | | 25% |
| 11 | B | Veegum HV | Stabilize | R. T. | 0.5% |
| | | magnesium aluminum | Emulsion | Vanderbilt | |
| | | silicate | | | |
| 12 | B2 | Sodium Chloride | Stabilizer | Brenntag | 0.70% |
| 13 | B2 | Sodium Citrate | Control | | 0.20% |
| | | | Acidity | | |
| 14 | B2 | Hyaluronic Acid 1% | Humectant | Rita Corp. | 0.10% |
| 15 | B2 | Aloe Vera Extract | Antioxidant | Active | 0.10% |
| | | | | Organics | |
| 16 | B2 | Chamomile Extract | Antioxidant | Active | 0.10% |
| | | | | Organics | |
| 17 | B2 | Euxyl 9010 | Cosmetic | Shin Etsu | 7.00% |
| | | phenoxyethanol | preservative | | |
| | | ethylhexylglyerin | | | |
| 18 | C | Zinclear IM | Sunscreen | Dow | 30.0% |
| | | Caprylic/carpric | | Corning | |
| | | Tiglyeride | | | |
| | | Glyceryl Isostearate | | | |
| | | Polyhydroxystearic | | | |
| | | acid | | | |
| | | | | | TOTAL |
| | | | | | 100.00% |

MANUFACTURING INSTRUCTIONS:

| | |
|---|---|
| PHASE | MIX PHASE A INGREDIENTS UNTIL |
| A | UNIFORM |
| PHASE | COMBINE PHASE B INGREDIENTS AND MIX UNTIL UNIFORM |
| B | |
| PHASE | SLOWLY ADD PHASE A TO PHASE B IN INCREMENTS. MAKE |
| B2 | SURE BATCH IS UNIFORM BEFORE ADDING NEXT INCREMENT |
| | MIX PHASE B2 UNTIL UNIFORM; COMBINE WITH PHASE A AND |
| | PHASE B |
| PHASE | ADD PHASES A, B AND B2 TO BATCH |
| C | AND MIX TO UNIFORMITY. |

TABLE 3A
PRODUCT CONTROL SPECIFICATION SPF 50 Mineral Lotion

| TEST | SPECIFICATION | RESULTS |
|---|---|---|
| APPEARANCE | OFF WHITE TO PALE | PASS - MATCH |
| | YELLOW LOTION | STD. |
| | BEIGE | |
| ODOR | TO MATCH STD. | PASS - MATCH |
| | | STD. |
| SPECIFIC GRAVITY | 1.0-1.24 | 1.113 |
| VISCOSITY | 18,000-80,000 CPS | 44,000 CPS |
| (SPINDLE TE/ | | |
| 10 RPM/1 MIN) | | |
| ASSAY: (15%) ZnO₂ | 13.5%-16.5% | 14.73% |
| ASSAY: (3.0%) TiO₂ | 2.7%-3.3% | 2.94% |
| BACTERIOLOGY | LESS THAN 10 CFU | PASS - LESS THAN |
| | MOLD/G, | 10 CFU - NO MOLD |
| | BACTERIA/G | AND NO |
| | NO PATHOGENS | PATHOGENS |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 2,798,053
U.S. Pat. No. 3,755,560
U.S. Pat. No. 4,421,769
U.S. Pat. No. 4,509,949
U.S. Pat. No. 4,599,379
U.S. Pat. No. 4,628,078
U.S. Pat. No. 4,835,206
U.S. Pat. No. 4,849,484
U.S. Pat. No. 5,011,681
U.S. Pat. No. 5,087,445
U.S. Pat. No. 5,100,660
U.S. Pat. No. 5,411,744
U.S. Pat. No. 5,871,761
U.S. Pat. No. 6,013,682
U.S. Pat. No. 6,203,802
U.S. Pat. No. 6,290,938
U.S. Pat. No. 6,387,398
The CTFA International Cosmetic Ingredient Dictionary, 4^{th} Ed., 1991.
Kreuter, J. Microencapsul., 5(2):115-27, 1988.
McCutcheon's, Detergents and Emulsifiers, North American Edition, 1986.
The CTFA International Cosmetic Ingredient Dictionary and Handbook, 12^{th} Ed., 2008.

What is claimed is:

1. A topically applicable cosmetic/dermatological UV-screening composition comprising:
(a) one or more metal oxides;
(b) an aesthetic enhancer including:
i) cross-linked silicone elastomers comprising dimethicone copolymer and dimethicone/vinyl dimethicone copolymer; and,
ii) at least one additional silicone compound,
wherein the aesthetic enhancer is effective for improving the aesthetic properties of the metal oxides;
(c) at least one antioxidant; and,
(d) a dermatologically acceptable vehicle.

2. The composition of claim 1, wherein the metal oxides are selected from the group consisting of titanium dioxide and zinc oxide.

3. The composition of claim 2, wherein the composition comprises from about 15.0% to about 17.0%, by weight, of zinc oxide and from about 1.5% to about 3.0%, by weight, of titanium dioxide.

4. The composition of claim 1, wherein it is formulated as a cream, a gel, an emulsion, a lotion, a solution, an anhydrous base, a stick, a spray, or an ointment.

5. The composition of claim 1, wherein the antioxidant is selected from the group consisting of camellia sinensis (green tea extract), punica granatum (pomegranate) extract, tetrahexyldecyl ascorbate (vitamin C), vaccinum macrocarpon (cranberry) fruit extract, and mixtures thereof.

6. The composition of claim 1, wherein the composition has a viscosity of about 50,000 to 200,000 centipoises.

7. The composition of claim 1, wherein the composition has a viscosity of about 18000 to 80000 centipoises.

8. The composition of claim 1, wherein the composition further comprises any one of the following:
(a) an emollient;
(b) an emulsifier;
(c) a preservative;
(d) a thickening agent;
(e) a structuring agent;
(f) a vitamin;
(g) a pharmaceutical ingredient;
(h) a hydrating agent, or
(i) a solvent
or a combination of ingredients (a) to (i).

9. The composition of claim 1, wherein the composition has an SPF value of at least 30.

10. The composition of claim 1, wherein the composition has an SPF value of at least 50.

11. The composition of claim 1, wherein the additional silicone compound is at least one or more linear silicone compounds.

12. The composition of claim 11, wherein the linear silicone compound is selected from the group consisting of dimethicone, caprylyl methicone, triethoxy caprylylsilane, and mixtures thereof.
